(19) United States
(12) Patent Application Publication
(10) Pub. No.: US 2006/0025318 A1
(43) Pub. Date: Feb. 2, 2006

(54) Composition for washing and conditioning keratin materials, comprising a carboxyalkyl starch, and process for the use thereof

(76) Inventors: Mireille Maubru, Chatou (FR); Béatrice Thomas, Issy les Moulineaux (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/111,710

(22) Filed: Apr. 22, 2005

Related U.S. Application Data
(60) Provisional application No. 60/566,411, filed on Apr. 30, 2004.

(30) Foreign Application Priority Data
Apr. 22, 2004 (FR) 04 50763

(51) Int. Cl. A61K 8/00

(52) U.S. Cl. 510/119

(57) ABSTRACT

The present disclosure relates to a composition for washing and conditioning keratin fibers, including, in a cosmetically acceptable medium: at least one anionic surfactant and at least one carboxymethyl starch. The disclosure also relates to the use of the composition for washing and conditioning keratin materials, and to a process for washing and conditioning keratin materials.

This application claims benefit of U.S. Provisional Application No. 60/566,411, filed Apr. 30, 2004, and French Patent Application No. 04 50763, filed Apr. 22, 2004, both of which are hereby incorporated by reference.

The present disclosure relates to a composition for washing and conditioning keratin materials, for example keratin fibers, wherein the composition comprises a washing base and a carboxymethyl starch. Further, the present disclosure relates to the use of the aforementioned composition for washing and conditioning keratin materials, such as keratin fibers.

In the field of conditioning shampoos, a washing base and a conditioning agent (which may be a cationic polymer, an amphoteric polymer, a silicone, a natural or synthetic oil or a fatty substance, or mixtures thereof), may be combined. These conditioning agents are used to improve the softness and disentangling of wet and dried hair, but may have a limited effect with respect to hair hold properties, and may have a tendency to make the hair lank and dull.

Washing compositions are often thickened, because it is desirable for washing products to be able to be measured out and taken up easily in the hand; accordingly, washing products must have a certain consistency or viscosity. Specifically, a liquid product is much more difficult to measure out and runs easily between the fingers.

Natural polymers such as celluloses are known to be useful for thickening washing compositions such as shampoos. Unfortunately, the use of cellulose thickeners may result in compositions that are unstable and/or are not smooth and homogeneous. Further, these thickeners may have the drawback of reducing the foam quality and the cosmetic performance of the shampoos, for example, by making the hair more laden and coarse. Also, the foam of such thickened compositions may not be not sufficiently mild and may not be easy to generate (either in terms of speed or abundance).

Thus, there is a need for a thickening system for suitably thickening washing compositions that either improves or does not affect the cosmetic and foaming properties of the washing compositions.

The use of carboxymethyl starches in cosmetics is also known. Specifically, patent application EP 1 188 432 describes the use of these starches as absorbing agents in solid cosmetic compositions.

The present inventors have discovered that, by using carboxymethyl starch in washing compositions, it is possible to overcome at least one of the drawbacks indicated above. Further, a stable composition of uniform and aesthetic appearance can be obtained, and the cosmetic properties, such as the smoothing power, the volumizing and the suppleness (malleability) of the hair, may be improved.

In one aspect, therefore, the present disclosure relates to a composition for washing and conditioning keratin materials, such as keratin fibers, comprising at least one anionic surfactant and at least one carboxymethyl starch.

The present disclosure also relates to the use of the aforementioned composition in a process for washing and conditioning keratin materials, such as keratin fibers, i.e. the hair.

The above aspects are not limiting. Other subjects, characteristics, aspects and advantages of the disclosure will become evident to one of ordinary skill upon reading the description and the various examples that follow.

The presently disclosed composition for washing keratin materials such as keratin fibers comprises, in a cosmetically acceptable medium:
from 3% to 50% by weight, relative to the total weight of the composition, of at least one anionic surfactant,
from 0.01% to 5% by weight, relative to the total weight of the composition, of at least one carboxyalkyl starch, and
from 60% to 97% by weight of water, relative to the total weight of the composition;
   wherein the weight ratio of the carboxyalkyl starch to the anionic surfactant ranges from 0.001 to 0.1.

For example, in one non-limiting embodiment, the weight ratio of the carboxyalkyl starch to the anionic surfactant ranges from 0.01 to 0.075.

Non-limiting examples of suitable carboxyalkyl starches include carboxy(C1-C4)alkyl starches, such as carboxymethyl starches.

The carboxyalkyl starches used herein may be pregelatinized. For the purposes of the present disclosure, the term “pregelatinized” means modified to significantly improve the swellability of the starch in cold (room temperature) water. Pregelatinization may be performed in the presence of water, by heat-treating the starch so as to split open some or all of the starch grains, thereby releasing the amylose and amylopectin therein. The release of amylose and amylopectin is accompanied by partial hydrolysis of the 1,4-glucoside bonds in the starch.

The carboxyalkyl starches that may be used herein include those derived from plants such as wheat, corn, rice, potato and cassaya (tapioca). In one non-limiting embodiment, the starch is derived from potato.

As used herein, the term “starches” refers to polysaccharides comprising a repetition of anhydroglucose units.

Carboxyalkyl starches may be obtained by grafting carboxyalkyl groups onto one or more of the alcohol functionalities of the starch. This grafting may be accomplished, for example, by the reaction of the starch with sodium monochloroacetate in an alkaline medium.

The carboxyalkyl groups may be attached to the starch via an ether function. For example, the carboxyalkyl groups may be attached via an ether functionality to carbon 1 of the starch.

In a non-limiting embodiment of the disclosure, the starch has a degree of substitution that ranges from 0.1 to 1. For the purpose of the present disclosure, degree of substitution is defined as corresponding to the mean number of hydroxyl groups substituted with an ester or ether group (in the present case ether for the carboxymethyl starches) per monosaccharide unit of the polysaccharide.

The carboxyalkyl starches may comprise units having the following formula: 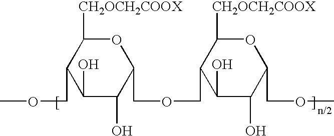
wherein X is chosen from a hydrogen atom, an alkali metal or alkaline-earth metal such as Na, K 1/2, Li, NH₄, a quaternary ammonium, or an organic amine. For example, X may represent an Na⁺ ion.

In a non-limiting embodiment of the disclosure, carboxyalkyl starches that may be used herein include pregelatinized carboxymethyl ether starches (for example, O-carboxymethyl starch), including those derived from potato, such as those sold under the name QUICKSOLAN CMS from the company Avebe.

The at least one carboxyalkyl starch disclosed herein may be present in the composition in an amount ranging from 0.01% to 5% by weight, for example from 0.05% to 3% by weight, such as from 0.1% to 2% by weight, relative to the total weight of the composition.

Non-limiting examples of suitable anionic surfactants that may be used in the presently disclosed composition include those chosen from salts, for example alkali metal salts, such as sodium salts; ammonium salts; amine salts; amino alcohol salts, or alkaline-earth metal salts, such as magnesium salts; these salts may be chosen from, for example: alkyl sulfates, alkyl ether sulfates, alkylamido ether sulfates, alkylarylpolyether sulfates, monoglyceride sulfates; alkyl sulfonates, alkylamide sulfonates, alkylaryl sulfonates, α-olefin sulfonates, paraffin sulfonates, alkyl sulfosuccinates, alkyl ether sulfosuccinates, alkylamide sulfosuccinates, alkyl sulfoacetates, acyl sarcosinates and acyl glutamates, wherein the alkyl and acyl groups of all these compounds contain from 6 to 24 carbon atoms, and the aryl group may be a phenyl or benzyl group.

Also useful here are monoesters of C₆₋₂₄ alkyl and of polyglycoside-dicarboxylic acids, such as, but not limited to, alkyl glucoside citrates, polyalkyl glycoside tartrates and polyalkyl glycoside sulfosuccinates, alkyl sulfosuccinamates, acyl isethionates and N-acyltaurates, wherein the alkyl or acyl group of all these compounds contains from 12 to 20 carbon atoms.

Another group of anionic surfactants that may be used in the compositions of the present disclosure include acyl lactylates, wherein the acyl group contains from 8 to 20 carbon atoms.

Further non-limiting examples of suitable anionic surfactants that may be used include alkyl-D-galactosiduronic acids and salts thereof; polyoxyalkylenated (C₆₋₂₄ alkyl)ethercarboxylic acids, polyoxyalkylenated (C₆₋₂₄ alkyl)(C₆₋₂₄ aryl)ether-carboxylic acids, polyoxyalkylenated (C₆₋₂₄ alkyl)amidoethercarboxylic acids, and salts thereof; and mixtures thereof. In one embodiment, for example, such anionic surfactants, comprising from 2 to 50 ethylene oxide units, may be used.

In a non-limiting embodiment of the present disclosure, alkyl sulfates, alkyl ether sulfates, alkyl ether carboxylates, and mixtures thereof may be used. In a further non-limiting embodiment, these compounds may be used in the form of an alkali metal, an alkaline-earth metal, an ammonium, an amine or an amino alcohol salt.

The at least one anionic surfactant may be present in the composition in a total amount ranging from 3% to 50% by weight, for example from 4% to 25% by weight, such as from 8% to 20% by weight relative to the total weight of the composition.

The composition may also comprise at least one nonionic surfactant and/or at least one amphoteric surfactant.

The total amount of washing base, i.e. the total amount of surfactants, in the composition, may range from 4% to 50% by weight, for example from 5% to 30% by weight, relative to the total weight of the composition.

As noted above, in a non-limiting embodiment, the weight ratio of the carboxyalkyl starch to the total amount of surfactants ranges from 0.001% to 0.1%, for example, from 0.01% to 0.075%.

Non-limiting examples of nonionic surfactants that may be used in the compositions of the present disclosure include compounds that are well known, such as those disclosed in the “Handbook of Surfactants,” by M. R. Porter, and published by Blackie & Son (Glasgow and London), 1991, pp. 116-178. For example, suitable nonionic surfactants include those chosen from polyethoxylated, polypropoxylated and polyglycerolated fatty acids, (C₁-C₂₀)alkylphenols, α-diols and alcohols having a fatty chain containing, for example, 8 to 18 carbon atoms. In various non-limiting embodiments, the number of ethylene oxide or propylene oxide groups can range from 2 to 50, and the number of glycerol groups can range from 2 to 30.

Further examples of nonionic surfactants include but are not limited to: condensates of ethylene oxide and of propylene oxide with fatty alcohols; polyethoxylated fatty amides, including those having from 2 to 30 ethylene oxide units, polyglycerolated fatty amides, including those containing on average from 1 to 5, such as from 1.5 to 4, glycerol groups; ethoxylated fatty acid esters of sorbitan, having from 2 to 30 ethylene oxide units; fatty acid esters of sucrose, fatty acid esters of polyethylene glycol, (C₆₋₂₄ alkyl)polyglycosides, N-(C₆₋₂₄ alkyl)glucamine derivatives, and amine oxides, such as (C₁₀₋₁₄alkyl)amine oxides and N—(C₁₀₋₁₄ acyl)aminopropylmorpholine oxides.

In non-limiting embodiment of the present disclosure, the at least one nonionic surfactant is a (C₆₋₂₄ alkyl)polyglycoside, such as a (C₈₋₁₈ alkyl)polyglycoside.

Non-limiting examples of the amphoteric surfactants that may be used in the present disclosure include: aliphatic secondary or tertiary amine derivatives in which the aliphatic group is a linear or branched chain containing from 8 to 22 carbon atoms and at least one anionic group, such as a carboxylate, sulfonate, sulfate, phosphate or phosphonate group. In addition, examples of suitable amphoteric surfactants include but are not limited to (C₈₋₂₀)alkylbetaines, sulfobetaines, (C₈₋₂₀)alkylamido(C₂₋₈alkyl)betaines and (C₈₋₂₀alkyl)amido(C₂₋₈alkyl)sulfobetaines.

Non-limiting examples of the aforementioned amine derivatives include the products sold under the name MIRANOL®, as described in U.S. Pat. Nos. 2,528,378 and 2,781,354 and classified in the CTFA dictionary, 3rd edition, 1982, under the names Amphocarboxyglycinate and Amphocarboxypropionate, having the respective structures (1) and (2):
Rₐ—CONHCH₂CH₂—N(R_{b})(R_{c})(CH₂COO⁻) (1)
wherein:
   Rₐ represents an alkyl group derived from an acid Rₐ—COOH present in hydrolysed coconut oil, a heptyl, nonyl or undecyl group,
   R_{b} represents a β-hydroxyethyl group, and
   R_{c} represents a carboxymethyl group; and
      Rₐ′—CONHCH₂CH₂—N(B)(Z) (2)
wherein:
   B represents CH₂CH₂OX′,
   Z represents —(CH₂)_{z}—Y′, with z=1 or 2,
   X′ represents a —CH₂CH₂—COOH group or a hydrogen atom,
   Y′ represents —COOH or a —CH₂—CHOH—SO₃H group, and
   Rₐ′ represents an alkyl group of an acid Rₐ′—COOH present in coconut oil or in hydrolysed linseed oil, a C₁₇ to C₂₃ saturated or unsaturated alkyl group, such as a C₁₇ alkyl group and its iso form, or an unsaturated C₁₇ group.

Non-limiting examples of these compounds include those that are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid.

Further non-limiting examples of suitable amine deriviatives include the cocoamphodiacetate products sold by the company Rhodia under the trade name MIRANOL® C2M concentrate.

In a non-limiting embodiment of the present disclosure, the amphoteric surfactants may be chosen from (C₈₋₂₀ alkyl)betaines, (C₈₋₂₀ alkyl)amido(C₂₋₈ alkyl)betaines, alkylamphodiacetates and mixtures thereof.

The nonionic and/or amphoteric surfactants may be present in the composition in an amount ranging from 0% to 20% by weight, for example from 0.5% to 10% by weight, such as from 1% to 5% by weight, relative to the total weight of the composition.

The composition may additionally comprise one or more cationic polymers. For the purposes of the present disclosure, the term “cationic polymer” denotes any polymer comprising cationic groups and/or groups that may be ionized into cationic groups.

Non-limiting examples of suitable cationic polymers that may be used in accordance with the present disclosure include those that are known for improving the cosmetic properties of hair, such as those described in European patent application EP-A-0 337 354, and in French patent applications FR-A-2 270 846, 2 383 660, 2 598 611, 2 470 596 and 2 519 863.

In a non-limiting embodiment, the cationic polymers to be used are those which contain units comprising primary, secondary, tertiary and/or quaternary amine groups that may form part of the main polymer chain or may be borne by a side substituent directly attached thereto.

The cationic polymers useful herein may have a number-average or weight-average molar mass ranging from 500 to 5×10⁶, such as from 10³ to 3×10⁶.

Among the cationic polymers useful herein, non-limiting mention may be made of polyamine, polyamino amide, and polyquaternary amide polymers

Non-limiting examples of suitable polyamine, polyamido amide and polyquaternary ammonium polymers that may be used in accordance with the present disclosure include those described in French patents Nos. 2 505 348 and 2 542 997. Among these polymers, non-limiting mention may be made of:
(1) homopolymers or copolymers derived from acrylic or methacrylic esters or amides and comprising at least one of the units of the following formulae: 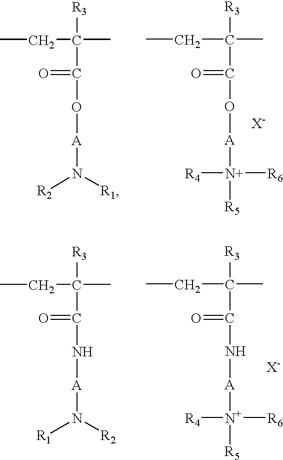
wherein:
R₃, which may be identical or different, is a hydrogen atom or a CH₃ radical;
A, which may be identical or different, represents a linear or branched alkyl group having from 1 to 6 carbon atoms, for example 2 or 3 carbon atoms, or a hydroxyalkyl group having from 1 to 4 carbon atoms;
R₄, R₅, R₆, which may be identical or different, are chosen from alkyl groups containing from 1 to 18 carbon atoms, for example an alkyl group containing from 1 to 6 carbon atoms, and from benzyl radicals;
R₁ and R₂, which may be identical or different, are chosen from hydrogen and alkyl groups containing from 1 to 6 carbon atoms, such as methyl or ethyl groups;
X is an anion derived from a mineral or organic acid, such as a methyl sulfate anion or a halide such as a chloride or bromide.

The copolymers of family (1) can also contain one or more units derived from comonomers which may be chosen from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Thus, among these copolymers of family (1), non-limiting mention may be made of:
copolymers of acrylamide and of dimethylaminoethyl methacrylate quaternized with dimethyl sulfate or with a dimethyl halide, such as those products sold under the name HERCOFLOC by the company Hercules,
the copolymers of acrylamide and of methacryloyloxyethyltrimethylammonium chloride, such as those that are described, for example, in patent application EPA080976 and sold under the name BINA QUAT P 100 by the company Ciba Geigy,
the copolymer of acrylamide and of methacryloyloxy-ethyltrimethylammonium methosulfate sold under the name RETEN by the company Hercules,
quaternized or non-quaternized vinylpyrrolidone/dialkylaminoalkyl acrylate or methacrylate copolymers, for example, the products sold under the name “GAFQUAT” by the company ISP, such as, for example, “Gafquat 734” or “Gafquat 755.”, or the products known as “Copolymer 845, 958 and 937,” which are described in detail in French patents 2 077 143 and 2 393 573
dimethylaminoethyl methacrylate/vinylcaprolactam/vinylpyrrolidone terpolymers, for example, the products sold under the name GAFFIX VC 713 by the company ISP,
vinylpyrrolidone/methacrylamidopropyldimethylamine copolymers, such as those sold under the name STYLEZE CC 10 by ISP,
and quaternized vinylpyrrolidone/dimethylaminopropylmethacrylamide copolymers, such as, for example, the product sold under the name “GAFQUAT HS 100” by the company ISP.

(2) cationic polysaccharides, for example, cationic celluloses and cationic galactomannan gums. Among the cationic polysaccharides, non-limiting mention may be made of cellulose ether derivatives comprising quaternary ammonium groups, cationic cellulose copolymers or cellulose derivatives grafted with a water-soluble quaternary ammonium monomer, and cationic galactomannan gums.

Non-limiting examples of suitable cellulose ether derivatives comprising quaternary ammonium groups (which are described in French patent 1 492 597) include polymers sold under the names “JR” (JR 400, JR 125, JR 30M) or “LR” (LR 400, LR 30M) by the company Amerchol. These polymers are also defined in the CTFA dictionary as hydroxyethylcellulose quaternary ammoniums that have reacted with an epoxide substituted with a trimethylammonium group.

Non-limiting examples of suitable cationic cellulose copolymers or cellulose derivatives grafted with a water-soluble quaternary ammonium monomer include those described in U.S. Pat. No. 4,131,576, for example, hydroxyalkylcelluloses, such as hydroxymethyl-, hydroxyethyl- or hydroxypropylcelluloses grafted especially with a methacryloylethyltrimethylammonium, a methacrylamidopropyltrimethylammonium or a dimethyldiallylammonium salt.

Commercial products corresponding to this description include the products sold under the names “CELQUAT L 200” and “CELQUAT H 100” by the company National Starch.

Of the aforementioned cationic galactomannan gums, non-limiting mention may be made of the compounds described in U.S. Pat. Nos. 3,589,578 and 4,031,307, including guar gums containing trialkylammonium cationic groups. Further, guar gums modified with a salt (e.g. chloride) of 2,3-epoxypropyltrimethylammonium may be used.

Suitable galactomannan gums include but are not limited to those sold under the trade names JAGUAR C13 S, JAGUAR C 15, JAGUAR C 17, and JAGUAR C162 by the company Rhodia.

(3) polymers consisting of piperazinyl units and of divalent alkylene or hydroxyalkylene radicals containing straight or branched chains, optionally interrupted by oxygen, sulfur or nitrogen atoms or by aromatic or heterocyclic rings, as well as the oxidation and/or quaternization products of these polymers. Such polymers include, but are not limited to, those described in French patents 2 162 025 and 2 280 361;

(4) water-soluble polyamino amides, including those prepared by the polycondensation of an acidic compound with a polyamine. These polyamino amides may be crosslinked with a crosslinking agent, such as an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative. The crosslinking agent may be, for example, used in proportions ranging from 0.025 to 0.35 mol per amine group of the polyamino amide. Further, these polyamino amides can be alkylated or, if they contain one or more tertiary amine functions, they can be quaternized. Non-limiting examples of such polymers include those described in French patents 2 252 840 and 2 368 508;

(5) polyaminoamide derivatives resulting from the condensation of polyalkylene polyamines with polycarboxylic acids followed by alkylation with difunctional agents. Non-limiting mention may be made, for example, of adipic acid/dialkylaminohydroxyalkyldialkylenetriamine polymers, wherein the alkyl radical contains from 1 to 4 carbon atoms (e.g., a methyl, ethyl or propyl radical). Such polymers are described in particular in French patent 1 583 363.

Among these derivatives, non-limiting mention may be made of the adipic acid/dimethylaminohydroxypropyl/diethylenetriamine polymers sold under the name “CARTARETINE F, F4 or F8” by the company Sandoz.

(6) polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms. The molar ratio between the polyalkylene polyamine and the dicarboxylic acid may range from 0.8:1 to 1.4:1. The polyamino amide that results is reacted with epichlorohydrin in a molar ratio of epichlorohydrin relative to the secondary amine group of the polyamino amide of 0.5:1 to 1.8:1. Such polymers are described, for example, in U.S. Pat. Nos. 3,227,615 and 2,961,347.

Examples of polymers of this type are sold under the name “HERCOSETT 57” by the company Hercules Inc., and under the name “PD 170” or “DELSETTE 101” by the company Hercules in the case of the adipic acid/epoxypropyl/diethylenetriamine copolymer.

(7) cyclopolymers of alkyldiallylamine or of dialkyldiallylammonium, for example, the homopolymers or copolymers containing, as a main constituent of the chain, units corresponding to formula (II) or (III): 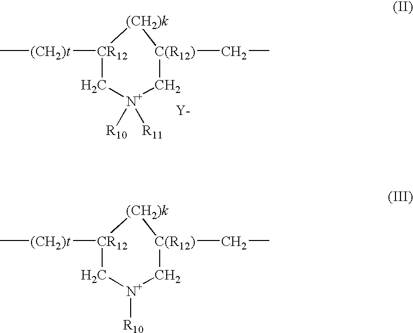
wherein k and t are equal to 0 or 1, the sum of k and t being equal to 1; R₁₂ is a hydrogen atom or a methyl radical; R₁₀ and R₁₁, independently of each other, are chosen from alkyl groups having from 1 to 6 carbon atoms, hydroxyalkyl groups in which the alkyl group has, for example, 1 to 5 carbon atoms, and lower (C₁-C₄) amidoalkyl groups; or, alternatively, R₁₀ and R₁₁ can form, together with the nitrogen atom to which they are attached, heterocyclic groups such as piperidyl or morpholinyl; Y⁻ is an anion, such as bromide, chloride, acetate, borate, citrate, tartrate, bisulfate, bisulfite, sulfate or phosphate. Non-limiting examples of suitable cyclopolymers of alkyldiallylamine or of dialkyldiallylammonium include those described in French patent 2 080 759 and in its Certificate of Addition 2 190 406.

In one non-limiting embodiment, R₁₀ and R₁₁, independently of each other, are chosen from alkyl groups containing from 1 to 4 carbon atoms.

Among the polymers defined above, non-limiting mention may be made of the dimethyldiallylammonium salt (chloride, for example) homopolymer sold under the name “MERQUAT 100” (and its low weight average molar mass homologues) by the company Nalco, and copolymers of diallyldimethylammonium salts (for example, chloride), and of acrylamide, such as those sold under the name “MERQUAT 550.”

(8) quaternary diammonium polymers containing repeating units corresponding to the formula: 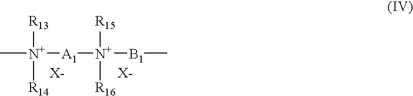
wherein:
R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals. Alternatively, R₁₃, R₁₄, R₁₅ and R₁₆, together or separately, may constitute, with the nitrogen atoms to which they are attached, heterocycles optionally containing a second hetero atom other than nitrogen. In addition, R₁₃, R₁₄, R₁₅ and R₁₆ may represent a linear or branched C₁-C₆ alkyl radical substituted with a nitrile, ester, acyl, or amide group, or a group —CO—O—R₁₇-D or —CO—NH—R₁₇-D, wherein R₁₇ represents an alkylene, and D is a quaternary ammonium group;
A₁ and B₁ represent linear or branched, saturated or unsaturated polymethylene groups containing from 2 to 20 carbon atoms, and also optionally containing, linked to or intercalated in the main chain, at least one aromatic ring or at least one atom chosen from oxygen and sulfur atoms, or at least one group chosen from sulfoxide, sulfone, disulfide, amino, alkylamino, hydroxyl, quaternary ammonium, ureido, amide and ester groups;
X⁻ is an anion derived from an inorganic or organic acid;
alternatively, A₁, R₁₃ and R₁₅ can form, with the two nitrogen atoms to which they are attached, a piperazine ring. Further, if A₁ is a linear or branched, saturated or unsaturated alkylene or hydroxyalkylene radical, B₁ can be a group (CH₂)ₙ—CO-D-OC—(CH₂)ₙ-wherein D is chosen from:
a) a glycol residue of formula: —O-Z-O—, where Z is a linear or branched hydrocarbon-based radical or a group corresponding to one of the following formulae:
   —(CH₂—CH₂—O)ₓ—CH₂—CH₂—
   —[CH₂—CH(CH₃)—O]_{y}—CH₂—CH(CH₃)—
where x and y are each integers from 1 to 4, representing a defined and unique degree of polymerization, or any number from 1 to 4, representing an average degree of polymerization;
b) a bis-secondary diamine residue, such as a piperazine derivative;
c) a bis-primary diamine residue of formula: —NH—Y—NH—, where Y is a linear or branched hydrocarbon-based radical, or alternatively the divalent radical
   —CH₂—CH₂—S—S—CH₂—CH₂—; and
d) a ureylene group of formula: —NH—CO—NH—;
In one non-limiting embodiment, X⁻ is an anion, such as chloride or bromide.

These quaternary ammonium polymers have a number-average molar mass generally ranging from 1000 to 100 000.

Non-limiting examples of suitable quaternary diammonium polymers include those described in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

In addtion to the above polymers, polymers having repeating units corresponding to the formula (V) may be used: 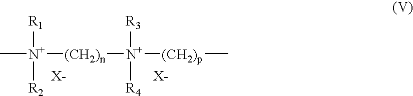
wherein R₁, R₂, R₃ and R₄, which may be identical or different, are chosen from alkyl or hydroxyalkyl radicals containing from 1 to 4 carbon atoms, n and p are integers ranging from approximately 2 to 20, and X⁻ is an anion derived from an inorganic or organic acid.

In one non-limiting embodiment, a compound of formula (V) known as Hexadimethrine chloride (per CTFA nomenclature), wherein R₁, R₂, R₃ and R₄ are methyl radicals, n=3, p=6 and X=Cl, is used.

(9) polyquaternary ammonium polymers comprising units of formula (VI): 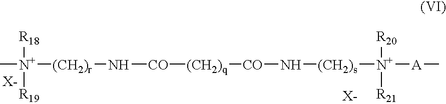
wherein:

R₁₈, R₁₉, R₂₀ and R₂₁, which may be identical or different, are chosen from a hydrogen atom, and from methyl, ethyl, propyl, β-hydroxyethyl, β-hydroxypropyl and —CH₂CH₂(OCH₂CH₂)ₚOH radicals,
where p is equal to 0 or an integer from 1 to 6, with the proviso that R₁₈, R₁₉, R₂₀ and R₂₁ do not simultaneously represent a hydrogen atom;
r and s, which may be identical or different, are integers from 1 to 6;
q is equal to 0 or to an integer from 1 to 34;
X⁻ is an anion such as a halide;
and A denotes a dihalide radical or —CH₂—CH₂—O—CH₂—CH₂—.

Non-limiting examples of these polyquaternary ammonium polymers are described, for example, in patent application EP-A-122 324.

Among these polyquaternary ammonium polymers, non-limiting mention may be made of, for example, the products “MRAPOL® A 15”, “MIRAPOL® AD1”, “MIRAPOL® AZ1” and “MIRAPOL® 175” sold by the company Miranol.

(10) quaternary polymers of vinylpyrrolidone and of vinylimidazole, such as the products sold under the names LUVIQUAT® FC 905, FC 550 and FC 370 by the company BASF.

(11) polyamines, such as POLYQUART® H sold by Cognis, which is listed in the CTFA dictionary under the name Polyethylene Glycol (15) Tallow Polyamine.

(12) optionally crosslinked methacryloyloxy(C₁-C₄)alkyltri(C₁-C₄)alkylammonium salt polymers, such as the polymers obtained by homopolymerization of dimethylaminoethyl methacrylate quaternized with methyl chloride, or by copolymerization of acrylamide with dimethylaminoethyl methacrylate quaternized with methyl chloride, wherein, if the polymer is crosslinked, the homo- or copolymerization is followed by crosslinking with a compound containing olefinic unsaturation, such as methylenebisacrylamide. In a non-limiting embodiment, a crosslinked acrylamide/methacryloyloxyethyltrimethylammonium chloride copolymer (20/80 by weight) in the form of a dispersion containing 50% by weight of the copolymer in mineral oil is used. A non-limiting example of this dispersion includes the product sold under the name “SALCARE®® SC 92” by the company Ciba. A dispersion containing crosslinked methacryloyloxyethyltrimethylammonium chloride homopolymer containing about 50% by weight of the homopolymer in mineral oil or in a liquid ester can also be used. Non-limiting examples of these dispersions include those sold under the names “SALCARE® SC 95” and “SALCARE® SC 96” by the company Ciba.

Other cationic polymers that can be used in the context of the present disclosure include cationic proteins or cationic protein hydrolysates, polyalkyleneimines such as polyethyleneimines, polymers containing vinylpyridine or vinylpyridinium units, condensates of polyamines and epichlorohydrin, quaternary polyureylenes and chitin derivatives.

In at least one non-limiting embodiment, the cationic polymers useful herein are chosen from quaternary cellulose ether derivatives such as the products sold under the name “JR 400” by the company Amerchol, cationic cyclopolymerssuch as the dimethyldiallylammonium salt (for example, chloride), homopolymers or copolymers sold under the names “Merquat 100”, “Merquat 550” and “Merquat S” by the company Nalco, and their low weight-average molecular weight homologues, quaternary polymers of vinylpyrrolidone and of vinylimidazole, optionally crosslinked homopolymers or copolymers of methacryloyloxy(C₁-C₄)alkyltri(C₁-C₄)alkylammonium salts, and mixtures thereof.

The cationic polymer(s) may be present in concentrations ranging from 0.01% to 20%, for example from 0.05% to 10%, such as from 0.1% to 5% by weight, relative to the total weight of the composition.

The composition according to the present disclosure may also comprise at least one silicone.

Non-limiting examples of suitable silicones that may be used in accordance with the present disclosure include polyorganosiloxanes that are insoluble in the composition, and may be in the form of oils, waxes, resins or gums.

The viscosity of the silicones is measured, for example, at 25° C. according to ASTM standard 445 Appendix C.

The above polyorganosiloxanes are disclosed in greater detail in Walter Noll's “Chemistry and Technology of Silicones” (1968) Academic Press. They can be volatile or non-volatile.

Among volatile silicones, non-limiting mention is made of silicones having a boiling point ranging from 60° C. to 260° C., for example:
(i) cyclic silicones containing from 3 to 7 silicon atoms, for example, from 4 to 5 silicon atoms. Non-limiting examples of these silicone include, for example, the octamethylcyclotetrasiloxane sold under the name “Volatile Silicone 7207” by Union Carbide or “Silbione 70045 V 2” by Rhodia, the decamethylcyclopentasiloxane sold under the name “Volatile Silicone 7158” by Union Carbide, “Silbione 70045 V 5” by Rhodia, and mixtures thereof.

Non-limiting mention may also be made of dimethylsiloxane/methylalkylsiloxane cyclo-copolymers, such as “Volatile Silicone FZ 3109” sold by the company Union Carbide, having the chemical structure:

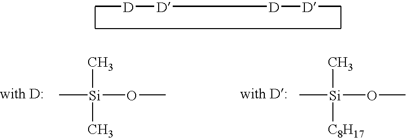

Non-limiting mention may also be made of mixtures of cyclic silicones with organosilicon compounds, such as a 50/50 mixture of octamethylcyclotetrasiloxane and tetratrimethylsilylpentaerythritol and a mixture of octamethylcyclotetrasiloxane and oxy-1,1′-bis(2,2,2′,2′,3,3′-hexatrimethylsilyloxy)neopentane; and
(ii) linear volatile silicones containing from 2 to 9 silicon atoms and having a viscosity of less than or equal to 5×10⁻⁶ m²/s at 25° C. A non-limiting example of a suitable linear volatile silicone is decamethyltetrasiloxane, which is sold under the name “SH 200” by the company Toray Silicone. Additional non-limiting examples of linear volatile silicones are described in Todd & Byers, “Volatile Silicone Fluids for Cosmetics,” Cosmetics and Toiletries, Vol. 91, Jan. 76, pp. 27-32.

In at least one non-limiting embodiment, a non-volatile silicone is used. The non-volatile silicone may be chosen from polyalkylsiloxanes, polyarylsiloxanes, polyalkylarylsiloxanes, silicone gums, silicone resins, polyorganosiloxanes modified with organofunctional groups, and mixtures thereof. These silicones are, for example, chosen from polyalkylsiloxanes, such as polydimethylsiloxanes containing trimethylsilyl end groups and having a viscosity of from 5×10⁻⁶ to 2.5 m²/s at 25° C., such as 1×10⁻⁵ to 1 m²/s.

Among the aforementioned polyalkylsiloxanes, non-limiting mention is made of the following commercial products:
the oils of the Mirasil series sold by the company Rhodia Chimie, such as MIRASIL DM 500 000;
the oils of the 200 series from the company Dow Corning, such as DC200 with a viscosity of 60 000 cSt;
the Viscasil oils and certain oils of the SF series (for example SF 96 and SF 18) from General Electric.

Non-limiting mention may also be made of polydimethylsiloxanes containing dimethylsilanol end groups (CTFA name: Dimethiconol), for example the oils of the 48 series from the company Rhodia Chimie.

In this category of polyalkylsiloxanes, mention may also be made of the products sold under the names “Abil Wax® 9800 and 9801” by the company Goldschmidt, which are poly(C₁-C₂₀)alkylsiloxanes.

The polyalkylarylsiloxanes, may, for example, be chosen from polydimethylmethylphenylsiloxanes, linear and/or branched polydimethyldiphenylsiloxanes with a viscosity of from 1×10⁻⁵ to 5×10⁻² m²/s at 25° C.

Among these polyalkylarylsiloxanes, non-limiting examples that may be mentioned include the products sold under the following names:
the Mirasil DPDM oils from Rhodia Chimie;
the oils of the Rhodorsil 70 633 and 763 series from Rhodia Chimie;
the oil Dow Corning 556 Cosmetic Grade Fluid from Dow Corning;
the silicones of the PK series from Bayer, for example, the product PK20;
the silicones of the PN and PH series from Bayer, for example, the products PN1000 and PH1000;
certain oils of the SF series from General Electric, for example SF 1023, SF 1154, SF 1250 and SF 1265.

The silicone gums that may be used in accordance with the disclosure include, for example, polydiorganosiloxanes having high number-average molecular masses ranging from 200,000 to 1,000,000, which may be used alone or in a mixture with a solvent. The solvent may be chosen from, but is not limited to volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Non-limiting mention may also be made of the following products:
polydimethylsiloxane,
polydimethylsiloxane/methylvinylsiloxane gums,
polydimethylsiloxane/diphenylsiloxane,
polydimethylsiloxane/phenylmethylsiloxane,
polydimethylsiloxane/diphenylsiloxane/methylvinylsiloxane.

Non-limiting examples of product mixtures that can be used in accordance with the present disclosure include:
mixtures formed from a polydimethylsiloxane hydroxylated at the chain end (known as dimethiconol according to the nomenclature of the CTFA dictionary), and from a cyclic polydimethylsiloxane (known as cyclomethicone according to the nomenclature of the CTFA dictionary), such as the product Q2 1401 sold by the company Dow Corning;
mixtures formed from a polydimethylsiloxane gum with a cyclic silicone, such as the product SF 1214 Silicone Fluid from the company General Electric. This product is an SF 30 gum corresponding to a dimethicone and has a number-average molecular weight of 500,000, dissolved in the oil SF 1202 Silicone Fluid corresponding to decamethylcyclopentasiloxane;
mixtures of two polydimethylsiloxanes of different viscosities, such as a mixture of a polydimethylsiloxane gum and a polydimethylsiloxane oil, for example, the product SF 1236 from the company General Electric. The product SF 1236 is a mixture of an SE-30 gum having a viscosity of 20 m²/s and an SF 96 oil, with a viscosity of 5×10⁻⁶ m²/s. This product may contain, for example, 15% SE 30 gum and 85% SF 96 oil.

The organopolysiloxane resins that can be used in accordance with the present disclosure include crosslinked siloxane systems containing the following units:

R₂SiO_{2/2}, R₃SiO_{1/2}, RSiO_{3/2} and SiO_{4/2}, wherein R represents a hydrocarbon-based group containing 1 to 16 carbon atoms or a phenyl group. Non-limiting examples of these products include those in which R is a C₁-C₄ lower alkyl radical, such as methyl, or is a phenyl radical.

Among these organopolysiloxane resins, non-limiting mention is made of the product sold under the name “Dow Corning 593” and the products sold under the names “Silicone Fluid SS 4230” and “Silicone Fluid SS 4267” by the company General Electric, which are silicones having a dimethyl/trimethyl siloxane structure.

Non-limiting mention may also be made of the trimethyl siloxysilicate type resins sold under the names X22-4914, X21-5034 and X21-5037 by the company Shin-Etsu.

The organomodified silicones that can be used in accordance with the disclosure include the silicones as defined above that contain one or more organofunctional groups attached via a hydrocarbon-based group.

Among these organomodified silicones, non-limiting mention may be made of polyorganosiloxanes comprising:
polyethyleneoxy and/or polypropyleneoxy groups optionally comprising C₆-C₂₄ alkyl groups, such as the products known as dimethicone copolyol sold by the company Dow Corning under the name DC 1248 or the oils Silwet L 722, L 7500, L 77 and L 711 by the company Union Carbide, and the (C₁₂)alkylmethicone copolyol sold by the company Dow Corning under the name Q2 5200;
substituted or unsubstituted amine groups, such as the products sold under the name GP 4 Silicone Fluid and GP 7100 by the company Genesee or the products sold under the names Q2 8220 and Dow Corning 929 or 939 by the company Dow Corning, for example, C₁-C₄ aminoalkyl groups;
thiol groups, such as the products sold under the names “GP 72 A” and “GP 71” from Genesee;
alkoxylated groups, such as the product sold under the name “Silicone Copolymer F-755” by SWS Silicones and Abil Wax 2428, 2434 and 2440 by the company Goldschmidt;
hydroxylated groups, such as the polyorganosiloxanes containing a hydroxyalkyl function, described in French patent application FR-A-85/16334;
acyloxyalkyl groups, such as the polyorganosiloxanes described in U.S. Pat. No. 4,957,732;
carboxylic anionic groups, such as, for example, in the products described in patent EP 186 507 from the company Chisso Corporation
alkylcarboxylic anionic groups, such as those present in the product X-22-3701E from the company Shin-Etsu; 2-hydroxyalkyl sulfonate; and 2-hydroxyalkyl thiosulfate, such as the products sold by the company Goldschmidt under the names “Abil S201” and “Abil S255”;
hydroxyacylamino groups, such as the polyorganosiloxanes described in patent application EP 342 834. Non-limiting mention may be made, for example, of the product Q2-8413 from the company Dow Corning.

According to the present disclosure, silicones comprising a polysiloxane portion and a portion consisting of a non-silicone organic chain may also be used, wherein one of the two portions constitutes the main chain of the polymer and the other portion is grafted onto the main chain. Non-limiting examples of these polymers are described, for example, in patent applications EP-A-412 704, EP-A-412 707, EP-A-640 105, WO 95/00578, EP-A-582 152 and WO 93/23009 and in U.S. Pat. Nos. 4,693,935, 4,728,571 and 4,972,037. These polymers are, in some embodiments, anionic or nonionic.

According to the present disclosure, the silicones can also all be used in the form of emulsions.

In at least one non-limiting embodiment, the silicones of the present disclosure may be chosen from:
non-volatile silicones selected from the family of polydimethylsiloxanes containing trimethylsilyl end groups, such as oils with a viscosity ranging from 0.2 to 2.5 m²/s at 25° C., for example, the oils of the DC200 series from Dow Corning, such as oils of the Mirasil DM series having a viscosity of 60,000 cSt, such as the oil Mirasil DM 500 000 sold by the company Rhodia Chimie or the silicone oil AK 300 000 from the company Wacker, polyalkylsiloxanes containing dimethylsilanol end groups such as dimethiconols, or polyalkylarylsiloxanes such as the oil Mirasil DPDM sold by the company Rhodia Chimie;
polydimethylsiloxanes containing amine groups such as amodimethicones or trimethylsilylamodimethicones.

For the purpose of the present disclosure, the term “cosmetically acceptable medium,” means a medium that is compatible with keratin materials, for example keratin fibers such as hair, but which also has a pleasant odor, appearance and feel. In at least one non-limiting embodiment, the medium is aqueous.

The cosmetically acceptable aqueous medium may comprise water or a mixture of water and at least one organic solvent chosen from C₁-C₄ lower alcohols, such as ethanol, isopropanol, tert-butanol or n-butanol; polyols such as glycerol, propylene glycol and polyethylene glycols.

In at least one non-limiting embodiment, the composition comprises from 70% to 95% by weight of water relative to the total weight of the composition.

The pH of the compositions according to the present disclosure is generally from 2 to 11, for example from 3 to 10, such as from 4 to 8.

The composition according to the present disclosure may further comprise additives such as anionic polymers other than carboxyalkyl starches, nonionic polymers, cationic polymers, amphoteric polymers, linear, branched or cyclic, volatile or non-volatile, organomodified or non-organomodified silicones, associative or non-associative polymeric thickeners, non-polymeric thickeners, foam synergists such as C₁₀-C₁₈ 1,2-alkanediols or fatty alkanolamides derived from mono- or diethanolamine derivatives of a C8-C24 fatty acid, nacreous agents, opacifiers, dyes or pigments, fragrances, mineral, plant or synthetic oils, waxes, fatty alcohols, oxyalkylenated fatty alcohols, vitamins, provitamins, UV-screening agents, free-radical scavengers, antidandruff agents, anti-seborrhoea agents, agents for preventing hair loss, preserving agents, pH stabilizers, solvents and mixtures thereof, and any other additives that are conventionally used in cosmetics and which do not affect the properties of the compositions according to the present disclosure.

The compositions in accordance with the present disclosure may also contain, in addition to the aforementioned combinations, viscosity regulators such as electrolytes, or thickeners (associative or non-associative thickeners). Non-limiting mention may also be made of sodium chloride, sodium xylenesulfonate, scleroglucans, xanthan gums, fatty acid alkanolamides, alkyl ether carboxylic acid, alkanolamides optionally oxyethylenated with up to 5 mol of ethylene oxide (such as the product sold under the name “Aminol A15” by the company Chem Y), crosslinked polyacrylic acids, and crosslinked acrylic acid/C₁₀-C₃₀ alkyl acrylate copolymers. Up to 10% by weight of these viscosity regulators may be used in the compositions according to the disclosure, relative to the total weight of the composition.

The compositions in accordance with the present disclosure may also contain, for example, up to 5% of nacreous agents or opacifiers that are well known in the prior art, such as, for example, C16 higher fatty alcohols (cetyl, stearyl or behenyl alcohol), fatty-chain acyl derivatives such as ethylene glycol or polyethylene glycol monostearate or distearate, and fatty-chain ethers, for instance distearyl ether or 1-(hexadecyloxy)-2-octadecanol.

A person skilled in the art will understand how to select the optional additives and the amount thereof such that the properties of the compositions of the present disclosure are not harmed.

The optional additives may be present in the composition according to the disclosure in an amount ranging from 0 to 20% by weight relative to the total weight of the composition.

The compositions according to the disclosure may be in the form of a liquid, for example, a liquid having a viscosity ranging from 10 to 6000 cps measured at 25° C. with a Rheomat RM 180 viscometer at a shear rate of 100 s⁻¹.

The compositions according to the disclosure may be in the form of an optionally thickened lotion, a cream, a mousse, or a gel. The compositions according to the disclosure are suitable for washing hair, but are not limited to such a use.

The compositions of the present disclosure may be packaged, for example, in bottles, pump-dispenser bottles, or in aerosol containers so as to enable application of the composition in vaporized form or in the form of a mousse. The compositions may also be impregnated in applicators, such as gloves or wipes.

The present disclosure also relates to a process for washing keratin materials, which comprises applying an effective amount of the composition as described above to the keratin materials, and then rinsing the keratin materials, for example with water, after an optional action time.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contain certain errors necessarily resulting from the standard deviation found in their respective testing measurements.

The examples below are non-limiting illustrations of the present disclosure. All of the amounts indicated represent percentage by weight, unless otherwise indicated.

### EXAMPLES

### Example 1

The following composition was prepared:

| Composition | Invention | | Comparative | |
|---|---|---|---|---|
| Sodium lauryl ether sulfate (70/30 | 14 | g AM | 14 | g AM |
| C12/C14) containing 2.2 mol of ethylene | | | | |
| oxide | | | | |
| Cocoylamidopropylbetaine | 1.9 | g AM | 1.9 | g AM |
| Diallyldimethylammonium chloride | 0.35 | g | 0.35 | g |
| homopolymer of MW 14 000 | | | | |
| Pregelatinized sodium carboxymethyl starch | 0.5 | g | — | |
| (Quicksolan CMS from the company Avebe) | | | | |
| Sodium carboxymethyl cellulose (Aquasorb | — | | 0.5 | g |
| A 500 from the company Aqualon) | | | | |
| Coconut acid monoisopropanolamide | 1 | g | 1 | g |
| Distearyl ether | 1.5 | g | 1.5 | g |
| Behenyl alcohol | 1.5 | g | 1.5 | g |
| Lauryl alcohol containing 2.5 mol of | 0.9 | g | 0.9 | g |
| ethylene oxide | | | | |
| Sodium chloride | 0.2 | g | 0.2 | g |
| Preserving agents, fragrance | qs | | qs | |
| Citric acid qs | pH 5.5 | | pH 5.5 | |
| Demineralized water qs | 100 | g | 100 | g |

*: Active Material (AM)

The inventive composition of example 1 gave a homogeneous and smooth appearance, in contrast with the comparative composition of example 1, which was granular. In other words, hair treated with the inventive composition was smoother than hair treated with the comparative composition.

### Examples 2 and 3

The following shampoo compositions were prepared:

| Composition | Example 2 | | Example 3 | |
|---|---|---|---|---|
| Sodium lauryl ether sulfate (70/30 | 15.4 | g AM | 15.5 | g AM |
| C12/C14) containing 2.2 mol of ethylene | | | | |
| oxide | | | | |
| Cocoyl betaine | 2.6 | g AM | 2.6 | g AM |
| Hydroxyethylcellulose crosslinked with | 0.4 | g | — | |
| epichlorohydrin and quaternized with | | | | |
| trimethylamine (JR 400 from the company | | | | |
| Amerchol) | | | | |
| Hydroxypropyltrimethylammonium guar | — | | 0.05 | g |
| chloride (Jaguar C30S by the company | | | | |
| Rhodia) | | | | |
| Pregelatinized sodium carboxymethyl | 1 | g | 1 | g |
| starch (Quicksolan CMS from the company | | | | |
| Avebe) | | | | |
| Polydimethylsiloxane of viscosity 500 000 | 0.5 | g | — | |
| cSt (Mirasil DM 500 000 from the company | | | | |
| Rhodia) | | | | |
| Polydimethylsiloxane of viscosity 300 000 | — | | 1 | g |
| cSt (Silicon Fluid AK 300 000 from the | | | | |
| company Wacker) | | | | |
| Coconut acid monoisopropanolamide | 0.5 | g | 1 | g |
| Mixture of 1-hexadecyloxyoctadodecanol | 2.5 | g | 2.5 | g |
| and of cetyl alcohol | | | | |
| Preserving agents, fragrance | qs | | qs | |
| Citric acid qs | pH 6.5 | | pH 6.3 | |
| Demineralized water qs | 100 | g | 100 | g |

Hair treated with the compositions of Examples 2 and 3 was smooth.

What is claimed is:

1. A composition for washing keratin materials, comprising, in a cosmetically acceptable aqueous medium:
from 3% to 50% by weight, relative to the total weight of the composition, of at least one anionic surfactant;
from 0.01% to 5% by weight, relative to the total weight of the composition, of at least one carboxyalkyl starch; and
from 60% to 97% by weight of water, relative to the total weight of the composition;
wherein the weight ratio of the carboxyalkyl starch to the anionic surfactant ranges from 0.001 to 0.1.

2. The composition of claim 1, wherein the at least one carboxyalkyl starch is a carboxy(C1-C4)alkyl starch.

3. The composition of claim 1, wherein the at least one carboxyalkyl starch is pregelatinized.

4. The composition of claim 1, wherein the at least one carboxyalkyl starch comprises units having the following formula: 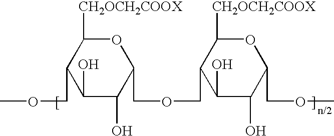
wherein X is chosen from a hydrogen atom, an alkali metal, an alkaline-earth metal, NH₄, a quaternary ammonium, and an organic amine.

5. The composition of claim 1, wherein the at least one carboxyalkyl starch is chosen from pregelatinized carboxymethyl ether starches

6. The composition of claim 5, wherein the at least one carboxyalkyl starch is a O-carboxymethyl starch.

7. The composition of claim 1, wherein the at least one carboxyalkyl starch is present in an amount ranging from 0.05% to 3% by weight relative to the total weight of the composition.

8. The composition of claim 1, wherein the at least one anionic surfactant is chosen from alkyl sulfates, alkyl ether sulfates, alkyl ether carboxylates, and mixtures thereof.

9. The composition of claim 1, wherein the at least one anionic surfactant is present in an amount ranging from 4% to 25% by weight relative to the total weight of the composition.

10. The composition of claim 1, further comprising at least one nonionic surfactant and/or at least one amphoteric surfactant.

11. The composition of claim 10, wherein the at least one nonionic surfactant is chosen from (C₆₋₂₄ alkyl)polyglycosides.

12. The composition of claim 10, wherein the at least one amphoteric surfactant is chosen from (C₈₋₂₀ alkyl)betaines, (C₈₋₂₀ alkyl)amido(C₂₋₈ alkyl)betaines, alkylamphodiacetates, and mixtures thereof.

13. The composition of claim 10, wherein the at least one nonionic and/or at least one amphoteric surfactant is/are present in a total amount ranging from 0 to 20% by weight relative to the total weight of the composition.

14. The composition of claim 13, wherein the at least one nonionic and/or at least one amphoteric surfactant is/are present in a total amount ranging from 0.5 to 10% by weight relative to the total weight of the composition.

15. The composition of claim 1, wherein the cosmetically acceptable aqueous medium comprises water, or a mixture of water with at least one organic solvent.

16. The composition of claim 15, wherein the at least one organic solvent is chosen from C₁-C₄ lower alcohols and polyols.

17. The composition of claim 1, further comprising at least one additional ingredient chosen from anionic polymers other than carboxyalkyl starches, nonionic polymers, cationic polymers, amphoteric polymers, linear, branched or cyclic, volatile or non-volatile, organomodified or non-organomodified silicones, associative or non-associative polymeric thickeners, non-polymeric thickeners, nacreous agents, opacifiers, dyes or pigments, fragrances, mineral, plant or synthetic oils, waxes, vitamins, provitamins, UV-screening agents, free-radical scavengers, antidandruff agents, anti-seborrhoea agents, agents for preventing hair loss, preserving agents, pH stabilizers, solvents and mixtures thereof.

18. The composition of claim 1, further comprising at least one cationic polymer.

19. The composition of claim 18, wherein the at least one cationic polymer is chosen from quaternary cellulose ether derivatives, cationic guar gums, cationic cyclopolymers, quaternary vinylpyrrolidone and vinylimidazole polymers, and mixtures thereof.

20. The composition of claim 18, wherein the at least one cationic polymer is chosen from homopolymers and copolymers of dimethyldiallylammonium salts.

21. The composition of claim 18, wherein the at least one cationic polymer is present in an amount ranging from 0.01% to 20% by weight relative to the total weight of the composition.

22. The composition of claim 21, wherein the at least one cationic polymer is present in an amount ranging from 0.05 to 10% by weight relative to the total weight of the composition.

23. The composition of claim 18, wherein the at least one cationic polymer is present in an amount ranging from 0.1 to 5% by weight relative to the total weight of the composition.

24. The composition of claim 1, further comprising at least one silicone.

25. A process for washing and conditioning keratin materials comprising
applying to a keratin material an effective amount of a composition comprising, in a cosmetically acceptable aqueous medium:
from 3% to 50% by weight, relative to the total weight of the composition, of at least one anionic surfactant;
from 0.01% to 5% by weight, relative to the total weight of the composition, of at least one carboxyalkyl starch; and
from 60% to 97% by weight of water, relative to the total weight of the composition;
wherein the weight ratio of the carboxyalkyl starch to the anionic surfactant ranges from 0.001 to 0.1, and
rinsing the keratin material after an optional action time.

26. The composition of claim 1, wherein said keratin materials are keratin fibers.

27. The process of claim 25, wherein said keratin materials are keratin fibers.
